(19) United States
(12) Patent Application Publication
(10) Pub. No.: US 2025/0228849 A1
(43) Pub. Date: Jul. 17, 2025

(54) FORMULATION AND MANUFACTURING PROCESS OF LIQUID FORMULATION OF RILPIVIRINE

(71) Applicant: Aptorum Therapeutics Limited, Hong Kong (HK)

(72) Inventor: Wai Yip Thomas LEE, Hong Kong (HK)

(21) Appl. No.: 19/023,112

(22) Filed: Jan. 15, 2025

Related U.S. Application Data
(60) Provisional application No. 63/620,884, filed on Jan. 15, 2024.

(51) Int. Cl. A61K 31/505; A61K 9/00; A61K 47/12; A61K 47/26; A61K 47/36; A61K 47/38

(52) U.S. Cl. CPC A61K 31/505; A61K 9/0053; A61K 47/12; A61K 47/26; A61K 47/36; A61K 47/38

(57) ABSTRACT

The present invention provides a manufacturing method of rilpivirine oral suspension with a specific loading of rilpivirine of 30-35 mg/mL. The oral suspension produced have a controlled pH and thixotropy, which are essential to facilitate the dissolution of rilpivirine hydrochloride into and even distribution and stability maintenance of the rilpivirine in the suspension, optimizing shelf life and efficacy of the suspension while also enabling convenient and palatable administration.

### CROSS-REFERENCE TO RELATED APPLICATION

The present application claims priorities to U.S. Provisional Patent Application No. 63/620,884, filed Jan. 15, 2024, the disclosures of each of which are incorporated herein by reference in their entireties.

### TECHNICAL FIELD

The present invention relates to a rilpivirine oral suspension formulation and manufacturing method thereof. More specifically, the present invention relates to the specific formulation and manufacturing for rilpivirine oral suspension specifically engineered to achieve certain thixotropy and pH value for optimized chemical stability, API solubility and administration convenience.

### BACKGROUND

Rilpivirine, including its hydrochloride salt form, is a second-generation non-nucleoside reverse transcriptase inhibitor (NNRTI), commonly used in human immunodeficiency virus type 1 (HIV-1) infection treatment.

The working mechanism of rilpivirine as an NNRTI is based on its inhibitory effect on the reverse transcriptase enzyme, through which the conversion process of viral RNA into DNA is prevented and thereby disrupting the HIV replication cycle.

Further, rilpivirine is designed in response to the resistance developed in certain HIV strains which exhibited resistance against earlier NNRTIs. Specifically, through binding to a specific site of the reverse transcriptase enzyme, rilpivirine effectively induces conformational changes to the enzyme and hence inhibiting its function.

With its enhanced potency and improved safety profile with fewer CNS-related adverse compared to other NNRTI like efavirenz, rilpivirine is becoming an increasing popular choice of HIV treatment medication, along with other drugs as a combination therapy.

Recently, rilpivirine is further used in anti-cancer therapy due to its efficacy also as an anti-tumour agent.

As of present, rilpivirine hydrochloride is available as film-coated tablets for oral administration, marketed under various trade names and long-acting injectables. However, oral formulations for rilpivirine hydrochloride remains underdeveloped.

This is due to rilpivirine having low aqueous solubility despite high permeability. Even in its hydrochloride salt form, which is an improvement to its solubility, rilpivirine hydrochloride is still categorized as a BCS Class II drug.

Moreover, rilpivirine and its other forms also require an acidic environment to optimize absorption.

Coupled with other factors including ease of administration, shelf life and palatability, an ideal rilpivirine formulation for oral administration is yet to be developed.

As such, while the chemical properties of rilpivirine in crystalline solid form is well studied, the demand for a stable and easily administrable rilpivirine oral formulation still remains unanswered. The present invention addresses this need.

### SUMMARY OF THE INVENTION

In view of the above, a manufacturing method of a rilpivirine oral suspension is provided herewith, the oral suspension having a specific loading of rilpivirine hydrochloride of 30-35 mg/mL, controlled pH and thixotropy for achieving an optimal drug stability and dose uniformity to ensure consistent therapeutic benefits.

The method comprises providing solid rilpivirine hydrochloride, subjecting the solid rilpivirine hydrochloride to physical micronization and obtaining a powder, mixing the micronized rilpivirine hydrochloride powder with purified water for a time period of at least 60 minutes to obtain a first suspension, mixing at least one thixotropic stabilizing agent, a preservative, a pH-adjusting buffer system and purified water and homogenizing under a speed of 5700-6200 RPM to obtain a second suspension, adding the first suspension to the second suspension and mixing for a time period of at least 60 minutes to receive a third suspension, and adding purified water to the third suspension to obtain the rilpivirine oral suspension.

Specifically, the rilpivirine oral suspension has a pH value of 1-3, and a thixotropic index of 4-10.

In an embodiment, the at least one thixotropic stabilizer is selected from xanthan gum, guar gum, tragacanth gum, carrageenan, microcrystalline cellulose, carboxymethylcellulose sodium, hydroxypropyl methylcellulose, and combinations thereof.

In a further embodiment, the at least one thixotropic stabilizer is xanthan gum, microcrystalline cellulose and carboxymethylcellulose sodium.

In yet another embodiment, the preservative is selected sodium benzoate.

In yet other embodiment, the pH-adjusting buffer system is selected from citrate buffer system comprising citric acid monohydrate and trisodium citrate dihydrate, phosphate buffer system comprising monosodium phosphate and disodium phosphate, or tartarate buffer system comprising tartaric acid monohydrate and sodium tartrate monohydrate.

In a further embodiment, the pH-adjusting buffer system is citrate buffer system comprising citric acid monohydrate and trisodium citrate dihydrate.

In a yet further embodiment, the second suspension is further mixed with a sweetener of concentration relative to the oral suspension of no more than 0.25%, and a flavoring of concentration relative to the oral suspension of no more than 0.6%.

In a second aspect of the invention, a rilpivirine oral suspension manufactured in accordance to the aforementioned method is also provided.

### BRIEF DESCRIPTION OF THE DRAWINGS

The disclosure will become more fully understood from the detailed description given herein below for illustration only, and thus not limitative of the disclosure, wherein:

FIG. 1 provides the chemical structure of the rilpivirine hydrochloride to be prepared into oral suspension in the present invention.

FIG. 2 provides a schematic illustration of the optimized manufacturing method of the exemplary formulation of the oral rilpivirine suspension of the present invention as discussed in the Example section below.

### DETAILED DESCRIPTION

In the following description, formulations and methods of manufacturing rilpivirine hydrochloride oral suspension are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Various processes will be described below to provide an example of at least one embodiment of the claimed subject matter. No embodiment described below limits any claimed subject matter, and any claimed subject matter may cover processes or systems that differ from those described below. The claimed subject matter is not limited to processes or systems having all of the features of any process or system described below or to features common to multiple or all of the processes or systems described below. It is possible that a process or system described below is not an embodiment of any claimed subject matter. Any subject matter that is disclosed in a process or system described below that is not claimed in this document may be the subject matter of another protective instrument, for example, a continuing patent application, and the applicants, inventors, or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures, and components have not been described in detail so as not to obscure the embodiments described herein. Also, the description is not to be considered as limiting the scope of the embodiments described herein.

It should be noted that terms of degree such as “substantially”, “about” and “approximately” as used herein mean a reasonable amount of deviation of the modified term such that the end result is not significantly changed. These terms of degree may be construed as including a certain deviation of the modified term if this deviation would not negate the meaning of the term it modifies.

Furthermore, the recitation of numerical ranges by endpoints herein includes all numbers and fractions subsumed within that. It is also to be understood that all numbers and fractions thereof are presumed to be modified by the term “about” which means a variation up to a certain amount of the number to which reference is being made if the end result is not significantly changed.

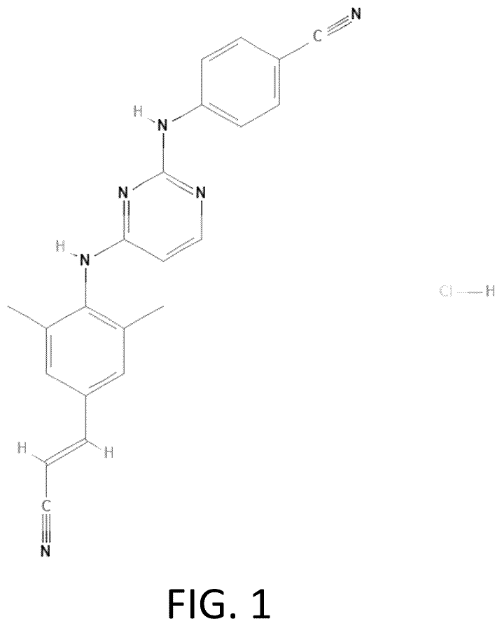

Rilpivirine hydrochloride, or 4-[[4-[[4-(2-cyanovinyl)-2,6-dimethylphenyl]amino]pyrimidin-2-yl]amino]benzonitrile hydrochloride in IUPAC nomenclature, is a second generation non-nucleoside reverse transcriptase inhibitor (NNRTI) with the molecular formula of C₂₂H₁₈N₆·HCl. Its chemical structure is shown in FIG. 1.

While rilpivirine hydrochloride is moderately soluble in methanol, ethanol and DMSO, its merely sparing solubility in water and its solubility dependency on pH (acidic media) makes it less favorable for oral formulation development.

As such, it poses a challenge in development of rilpivirine oral formulation that the potential non-uniform dispersion of rilpivirine hydrochloride in the formulation due to its poor water solubility must be overcome through stabilizing agents.

To start with, solid rilpivirine hydrochloride is further reduced to a particular size of micrometer range, typically under 10 μm, through micronization process. Particularly, for the present invention, as the rilpivirine hydrochloride is micronized through mechanical processes including but not limited to jet milling or ball milling.

However, the physiochemical and pharmacokinetic properties of rilpivirine hydrochloride must also be taken into consideration such that the oral formulation. Even if irreversible sedimentation and aggregation (“hard caking”) is prevented and relatively uniform dispersion of rilpivirine hydrochloride in the oral suspension is achieved, the formulation must still be of a reasonably low level of viscosity to facilitate subject intake.

This is achievable by using thixotropic excipients. Thixotropy refers to a time-dependent shear-thinning property, where the viscosity of a fluid or semi-solid decreases under shear stress (e.g. stirring or shaking) but gradually regains viscosity upon removal of the stress. In other words, thixotropic behavior allows a material to flow more easily when agitated, and when at rest the original consistency is restored.

The measurement of thixotropicity is by thixotropic index, which is defined the ratio of viscosity at low shear rates to viscosity at high shear rates, with the equation as below:

$$Thixotropic Index = \frac{Viscosity at Low Shear Rate}{Viscosity at High Shear Rate}$$

Therefore, a lower thixotropic index indicates easier flow upon agitation and resuspension thereafter; whereas a higher thixotropic index indicates more stable suspensions and slower sedimentation at rest.

The rilpivirine oral suspension of the present invention is formulated to have a thixotropic index of 4-10, optimal in balancing the stability of suspension which equates better maintenance of chemical stability at rest and longer shelf life, and the ease of administration due to easier flow upon agitation for easier uptake by patients.

Thixotropic excipients suitable for the rilpivirine oral formulation of the present invention are xanthan gum, guar gum, tragacanth gum, carrageenan, microcrystalline cellulose, carboxymethylcellulose sodium, hydroxypropyl methylcellulose. These excipients are also intrinsically able to act as anti-caking agents, facilitating even dispersion of the rilpivirine in the formulation.

Rilpivirine hydrochloride also has a pKₐ of approximately 5.6 for the pyrimidine nitrogen. This in turn affects its ionization and solubility to be more soluble in acidic media. To achieve the acidic environment to promote the dissolution of rilpivirine hydrochloride in aquatic medium, a pH-adjusting buffer system is also included in the oral formulation of the present invention.

Buffer systems that possess pH-adjusting capabilities, particularly functionalized for acidic environments to enhance water solubility of rilpivirine hydrochloride, include citrate buffer system comprising citric acid monohydrate and trisodium citrate dihydrate, phosphate buffer system comprising monosodium phosphate and disodium phosphate, or tartarate buffer system comprising tartaric acid monohydrate and sodium tartrate monohydrate.

Additionally, a preservative is also included in the formulation to enhance antimicrobial efficacy.

Optionally, due to the bitter taste of the rilpivirine hydrochloride when dissolved in water, sweetener and flavor can be added to the oral formulation for better palatability.

As such, an oral suspension formulation with rilpivirine hydrochloride as active pharmaceutical ingredient (API) with a specific loading of 30-40 mg/mL is provided herewith in this present invention, striking a balance between considerable shelf life, chemical stability, ease of administration and effective API dissolution and uniform dispersion for optimal drug efficacy.

### Examples

In the Example section below, the optimum manufacturing method and formulation for the rilpivirine oral suspension/in particular is discussed in detail.

The materials used for the development of the exemplary formulation of the rilpivirine oral suspension of the present invention, with a specific loading of 33 mg/mL, is tabulated in Table 1 below.

TABLE 1
Exemplary formulation composition of the
rilpivirine hydrochloride oral suspension

| | | Concentration | |
|---|---|---|---|
| Component | Function | (% w/v) | mg/mL |
| Rilpivirine hydrochloride (API) | Active | 3.30 | 33 |
| Microcrystalline Cellulose and | Suspending | 0.38 | 3.8 |
| Carboxymethylcellulose | Agent | | |
| Sodium, USP-NF | | | |
| Xanthan Gum, USP-NF | Suspending | 0.10 | 1 |
| | Agent | | |
| Sodium benzoate, USP-NF | Preservative | 0.50 | 5 |
| Cherry flavor | Flavor | 0.50 | 5 |
| Citric acid monohydrate, | Buffer | 0.35 | 3.5 |
| USP-NF | | | |
| Trisodium citrate dihydrate, | Buffer | 0.30 | 3 |
| USP-NF | | | |
| Sucralose, USP-NF | Sweetener | 0.20 | 2 |
| Purified water USP | Solvent | QS to 100% | QS to |
| | | | 1 mL |

API = active pharmaceutical ingredient;
NF = National Formulary;
QS = quantity sufficient;
USP = United States Pharmacopeia

Specifically, a combination of microcrystalline cellulose, carboxymethylcellulose sodium and xanthan gum is used as the thixotropic stabilizing agent to induce thixotropy to the oral formulation while facilitating the uniform dispersion of rilpivirine hydrochloride in the formulation. As such, the thixotropic index of the formulation is controllable within a range of 4-10.

Citrate buffer system, comprising citric acid monohydrate and trisodium citrate dihydrate, is used in the exemplary formulation as the pH-adjusting buffer system. The citrate buffer system is capable of adjusting the pH value of the formulation at a range of 1-3, which is crucial for the dissolution of rilpivirine hydrochloride into the suspension due to its physiochemical properties, as discussed above.

The bitter taste of dissolved rilpivirine hydrochloride is taken into consideration and counter-balanced in this exemplary oral formulation through the incorporation of sucralose sweetener and cherry flavor. Note that the sweetener and flavor sums up to no more than 0.85% w/v of the formulation, to ensure the thixotropy and viscosity of the oral suspension is maintained.

The preservative utilized in this oral suspension formulation is sodium benzoate, which is also effective under acidic environments, therefore a suitable choice for rilpivirine hydrochloride suspension which requires acidic media.

With the engineered and formulated w/v concentrations of the components discussed above calculated, the remainder of the formulation is made up by purified water of quantum satis.

This exemplary rilpivirine oral suspension is designed to have a pH value of 1-3, favorable for rilpivirine hydrochloride dissolution, and a thixotropic index of 4-10, where the viscosity at rest allows the rilpivirine hydrochloride suspension to settle and maintain chemical stability; while the viscosity upon agitation (by, for example, shaking) is lower for easier administration and uptake by patient.

With the stabilizing agents, relatively uniform dispersion of rilpivirine hydrochloride as API is achieved, thereby allowing precise dosage administration by calculating with the specified API loading of the oral suspension.

The sweetener and flavor also enhance palatability and improves patient experience when administering the rilpivirine dosage orally.

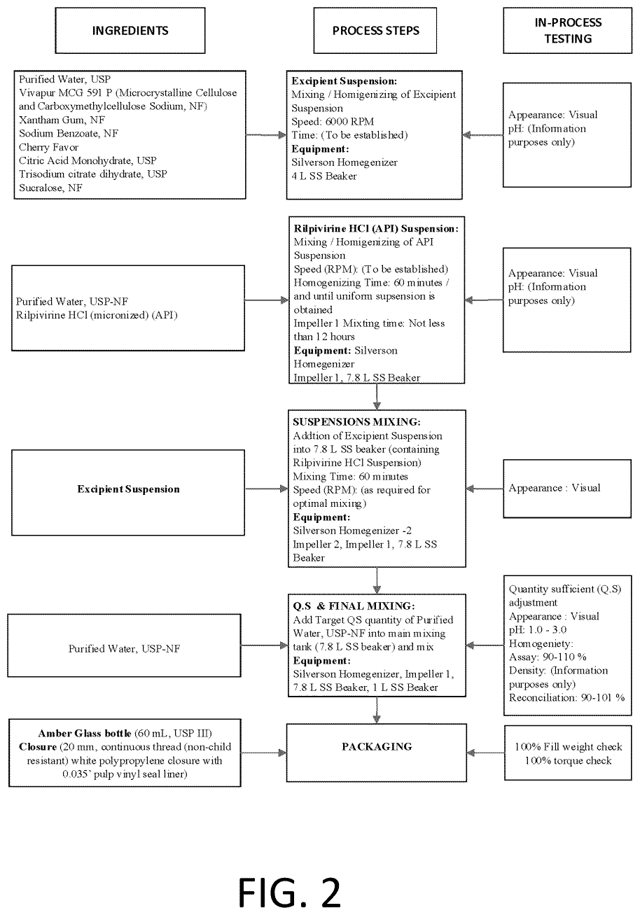

Further, with reference to the schematic diagram of manufacturing process in FIG. 2, the oral suspension with the exemplary formulation above can be manufactured without complicated processes and cutting-edge technologies or apparatuses, which grants the rilpivirine oral suspension of the present invention high scalability, and consequently immense potential for large-scale production and industrial applicability.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

1. A method for manufacturing rilpivirine oral suspension, the oral suspension having a specific loading of rilpivirine hydrochloride of 30-40 mg/mL, controlled pH and thixotropy for achieving an optimal drug stability and dose uniformity to ensure consistent therapeutic benefits, wherein the method comprises:
providing solid rilpivirine hydrochloride;
subjecting the solid rilpivirine hydrochloride to physical micronization and obtaining a powder;
mixing the micronized rilpivirine hydrochloride powder with purified water for a time period of at least 60 minutes to obtain a first suspension;
mixing at least one thixotropic stabilizing agent, a preservative, a pH-adjusting buffer system and purified water and homogenizing under a speed of 5700-6200 RPM to obtain a second suspension;
adding the first suspension to the second suspension and mixing for a time period of at least 60 minutes to receive a third suspension; and
adding purified water to the third suspension to obtain the rilpivirine oral suspension;
wherein the pH value of the rilpivirine oral suspension is 1 to 3;
wherein the rilpivirine oral suspension has a thixotropic index of 4-10.

2. The method of claim 1, wherein the at least one thixotropic stabilizer is selected from xanthan gum, guar gum, tragacanth gum, carrageenan, microcrystalline cellulose, carboxymethylcellulose sodium, hydroxypropyl methylcellulose, and combinations thereof.

3. The method of claim 2, wherein the at least one thixotropic stabilizer is xanthan gum, microcrystalline cellulose and carboxymethylcellulose sodium.

4. The method of claim 1, wherein the preservative is selected sodium benzoate.

5. The method of claim 1, wherein the pH-adjusting buffer system is selected from citrate buffer system comprising citric acid monohydrate and trisodium citrate dihydrate, phosphate buffer system comprising monosodium phosphate and disodium phosphate, or tartarate buffer system comprising tartaric acid monohydrate and sodium tartrate monohydrate.

6. The method of claim 5, wherein the pH-adjusting buffer system is citrate buffer system comprising citric acid monohydrate and trisodium citrate dihydrate.

7. The method of claim 1, wherein:
the concentration of rilpivirine hydrochloride relative to the oral suspension is 3.0-3.5% w/v;
the total concentration of the thixotropic stabilizing agents relative to the oral suspension is 0.4-0.5% w/v;
the total concentration of the buffer system components relative to the oral suspension is 0.5-0.7% w/v; and
the concentration of the preservative relative to the oral suspension is 0.4-0.6% w/v.

8. The method of claim 7, wherein the second suspension is further mixed with a sweetener of concentration relative to the oral suspension of no more than 0.25%, and a flavoring of concentration relative to the oral suspension of no more than 0.6%.

9. The method of claim 8, wherein the sweetener is sucralose.

10. A rilpivirine oral suspension manufactured in accordance to the method of claim 9.
